(19) United States
(12) Patent Application Publication
(10) Pub. No.: US 2025/0258161 A1
(43) Pub. Date: Aug. 14, 2025

(54) COMPLEMENT-DEPENDENT CYTOTOXICITY METHODS

(71) Applicant: AMGEN INC., Thousand Oaks, CA (US)

(72) Inventors: Andrew W. HORTON, Cambridge, MA (US); Kristin R. ABRAMS, Reading, MA (US); Jill A. CROUSE-ZEINEDDINI, Newbury Park, CA (US)

(73) Assignee: AMGEN INC., Thousand Oaks, CA (US)

(21) Appl. No.: 18/855,457

(22) PCT Filed: Apr. 13, 2023
(86) PCT No.: PCT/US2023/018527

§ 371 Date: Oct. 9, 2024

Related U.S. Application Data
(60) Provisional application No. 63/331,059, filed on Apr. 14, 2022.

(51) Int. Cl. G01N 33/50

(52) U.S. Cl. CPC G01N 33/5014; G01N 2333/4716

(57) ABSTRACT

The present invention relates to complement-dependent cytotoxicity assays and methods of addition of complement to cells.

### CROSS-REFERENCE TO RELATED APPLICATIONS

This application claims the benefit of U.S. Provisional Application No. 63/331,059, filed Apr. 14, 2021, which is hereby incorporated by reference in its entirety.

### FIELD OF THE INVENTION

The present invention relates to cytotoxicity methods. In particular, the present invention relates to complement-dependent cytotoxicity methods, and methods to reduce the variability of complement-cytotoxicity assays.

### BACKGROUND OF THE INVENTION

The complement system is a part of the immune system that is comprised of a number of different proteins that function to attack a pathogen's cell membrane. The classical complement cascade pathway can be initiated by the binding of the complement (e.g. C1q) protein to IgG antibodies. Complement Dependent Cytotoxicity (“CDC”) assays are used to measure complement cascade activation for many molecules such as biopharmaceuticals. These assays can be used for characterization of the ability of the product to activate the complement cascade to avoid undesired safety concerns or these can be used to measure the mechanism of activation of the targeted pathway.

CDC assay methods are well known. For example, see e.g. Duensing and Watson, Cold Spring Harb Protoc; 2018 Feb. 1; 2018 (2). The classical approach for CDC is to add serum comprising the components of the complement system to target cells bound by the antibody being tested, and then to determine either cell membrane integrity via use of cells preloaded with dyes, or to determine cell death. Various sources of complement or tissue culture grade serum are commercially available from multiple vendors, including Cedarlane®, EMD Millipore, Sigma Aldrich, and Bio-Rad.

When CDC assays are used as part of assays such as product release, controlling variability is imperative. However, the inventors of the present invention determined that the CDC assay may exhibit variability and that slower speeds of complement addition contributed to this variability by causing non-specific cytotoxicity. This non-specific cytotoxicity before the addition of product results in undesirable assay variability. While CDC assay methods are well known, Duensing and Watson (Cold Spring Harb Protoc; 2018 Feb. 1; 2018 (2)), for example, describes a CDC method but does not describe the speed of complement addition. Wang et al. also describe a CDC assay, however to reduce antibody-independent nonspecific cytotoxicity the assay includes an additional step of pre-absorbing the complement on target cells for 20 minutes (MAbs. 2020 January-December; 12(1): 1690959).

The present invention seeks to solve the problem of non-specific cytotoxicity (and thereby method variability) by increasing the speed of addition of baby rabbit complement addition in the CDC assay. The non-specific killing of target cells is reduced to an acceptable level if the complement is added to the cells at a faster speed. In addition, alternatively, non-specific cytotoxicity is reduced if the complement is added to the assay plate before the addition of the target cells. These improved methods result in less assay variability leading to a more robust method.

### SUMMARY OF THE INVENTION

The present invention provides methods of performing a cytotoxicity assay. In an embodiment, the present invention provides methods of performing a complement-dependent cytotoxicity (CDC) assay. In an embodiment, the method comprises adding complement to a mixture of target cells and test molecule, wherein the complement is added at a speed of at least about 250 μL/s. In a particular embodiment, the complement is added at a speed of about 250 μL/s. In a particular embodiment, the complement is added at a speed of about 300 μL/s. In a particular embodiment, the complement is added at a speed of about 350 μL/s. In a particular embodiment, the complement is added at a speed of at least about 400 μL/s. In another particular embodiment, the complement is added at a speed of about 400 μL/s. In another particular embodiment, the complement is added at a speed of about 450 μL/s. In yet another particular embodiment, the complement is added at a speed of about 500 μL/s.

In an embodiment, the method comprises adding complement to a mixture of target cells and test molecule, wherein the complement is added at a speed of at least 250 μL/s. In a particular embodiment, the complement is added at a speed of 250 μL/s to 500 μL/s. In a particular embodiment, the complement is added at a speed of 250 μL/s. In a particular embodiment, the complement is added at a speed of 300 μL/s. In a particular embodiment, the complement is added at a speed of 350 μL/s. In a particular embodiment, the complement is added at a speed of at least 400 μL/s. In another particular embodiment, the complement is added at a speed of 400 μL/s. In another particular embodiment, the complement is added at a speed of 450 μL/s. In yet another particular embodiment, the complement is added at a speed of 500 μL/s.

In an embodiment, the complement is baby rabbit complement. In a particular embodiment, the complement is from Bio-Rad or Cedarlane®. In a particular embodiment, the complement is not from EMD.

The present invention also provides a method of performing a CDC assay comprising adding target cells and test molecule to a mixture comprising complement and media. In an embodiment, the target cells are added to the complement and media mixture, followed by addition of test molecule. In an embodiment, the target cells and test molecule are added at the same time to the complement and media mixture. In an embodiment, the complement is baby rabbit complement. In an embodiment, the complement is human complement. In a particular embodiment, the complement is from Bio-Rad, Cedarlane®, or EMD.

The present invention also provides a method of adding complement to target cells, comprising adding complement to the target cells at a speed of at least about 250 μL/s. In an embodiment, the complement is added to the target cells at a speed of at least about 400 μL/s. In an embodiment, the complement is added to the target cells at a speed of about 250 μL/s. In an embodiment, the complement is added to the target cells at a speed of about 300 μL/s. In an embodiment, the complement is added to the target cells at a speed of about 350 μL/s. In an embodiment, the complement is added to the target cells at a speed of about 400 μL/s. In an embodiment, the complement is added to the target cells at a speed of about 450 μL/s. In an embodiment, the complement is added to the target cells at a speed of about 500 μL/s. In an embodiment, the complement is added to the target cells at a speed of at least about 500 μL/s.

In a particular embodiment, the complement is added at a speed of 250 μL/s to 500 μL/s. In an embodiment, the complement is added to the target cells at a speed of at least about 400 μL/s. In an embodiment, the complement is added to the target cells at a speed of about 250 μL/s. In an embodiment, the complement is added to the target cells at a speed of about 300 μL/s. In an embodiment, the complement is added to the target cells at a speed of about 350 μL/s. In an embodiment, the complement is added to the target cells at a speed of about 400 μL/s. In an embodiment, the complement is added to the target cells at a speed of about 450 μL/s. In an embodiment, the complement is added to the target cells at a speed of about 500 μL/s. In an embodiment, the complement is added to the target cells at a speed of at least about 500 μL/s.

In an embodiment, the complement is baby rabbit complement. In an embodiment, the complement is from Bio-Rad or Cedarlane. In an embodiment, the complement is not from EMD. In an embodiment, the complement is added to target cells in an assay to determine cell death. In an embodiment, the assay is a complement-dependent cytotoxicity assay. In an embodiment, the complement is baby rabbit complement. In a particular embodiment, the complement is from Bio-Rad or Cedarlane®. In a particular embodiment, the complement is not from EMD.

In an embodiment, the test molecule is an IgG1 or IgG3 molecule.

The present invention also provides a method performing a CDC assay (Assay 1), wherein if undesired non-specific cell killing is observed in Assay 1, the CDC assay is subsequently performed (Assay 2) in a method comprising an increase in the speed of complement addition. The present invention also provides a method performing a CDC assay (Assay 1), wherein if undesired non-specific cell killing is observed, the CDC assay is subsequently performed (Assay 2) in a method comprising adding target cells and test molecule to a mixture comprising complement and media. In an embodiment, there is a reduction in non-specific cell killing in Assay 2 compared to the non-specific cell killing in Assay 1.

### BRIEF DESCRIPTION OF THE DRAWINGS

FIG. 1. Effect of the speed of addition of different sources of Baby Rabbit Complement (BRC) on the control ratio. “Control (Media)” refers to wells having cells and media. “Cells+Complement” refers to wells having cells, media, and complement. The speed of complement (or media alone) is depicted in the legend, and is either 10 μL/second, 50 μL/second, 100 μL/second, 200 μL/second, 400 μL/second, or a manual addition at 429 μL/second. * indicates a statistically significant p-value <0.0001, and “ns” indicates not statistically significant.

FIG. 2. Luminescence demonstrating nonspecific killing of WIL2-S cells is increased when cells are added before addition of BRC compared to when BRC is added before addition of cells (“Cells+BRC” compared to “BRC+Cells”).

### DETAILED DESCRIPTION

The present invention provides improved methods for reducing variability in cytotoxicity assays, for example, by reducing the amount of non-specific cell killing observed in cytotoxicity assays. In particular, the present invention provides complement-dependent cytotoxicity methods with reduced non-specific cell killing.

A cytotoxicity assay refers to an assay that determines cell death. As used herein, “cell death” may be used interchangeably with “cell killing” or “cell lysis.” “Complement-dependent cytotoxicity” refers to cell killing observed in the CDC assay. In a CDC assay, when target cells, test molecule, and complement interact, membrane attack complex is formed on the target cell's surface, after which the target cells undergo cell lysis. A CDC assay determines the amount of cell lysis.

As used herein, a “complement-dependent cytotoxicity assay” or “CDC assay” refers to an assay used to measure killing of target cells that have been mixed with complement and a test molecule, such as for example in media in a cell culture plate. In some aspects as described herein, a CDC assay is performed without adding test molecule, so as to determine the amount of non-specific cell killing. Non-specific cell killing refers to cell death in the absence of test molecule.

A CDC assay can be performed by methods widely known by a person of ordinary skill, including modified methods. For example, a CDC assay can be performed by adding cells expressing a target to a plate in media, and then adding complement (such as baby rabbit complement) and a test molecule that bind the target. If the test molecule is an antibody having an Fc, for example, the antibody binds the target expressed on the cells and the Fc portion of the antibody binds complement. Binding complement initiates a complement cascade, resulting in death of the cell expressing the target. The present invention provides improved methods of cytotoxicity assays by adding complement at a higher speed. Such higher speed of complement addition results in reduced non-specific cell killing and increased method precision and accuracy.

A CDC assay of the present invention is also performed by a method comprising adding target cells and test molecule to a mixture comprising complement and media. In such methods, complement and media are first added to a plate, and then target cells and test molecule are added to the plate. In this particular method, the speed of complement addition is not critical to reduce non-specific cell killing.

Unless defined otherwise, all technical and scientific terms used herein have the same meaning as commonly understood by one of ordinary skill in the art to which this invention belongs.

As used herein, a “plate” is used interchangeably with “well” of a plate, flask, or other material that is suitable for cell culture.

A CDC assay may be performed at a time and temperature that is suitable for the target cell type and test molecule.

As used herein, the term “about,” when used in reference to a particular recited numerical value, means that the value may vary from the recited value by no more than 10%.

A CDC assay may be performed according to known methods and improved methods described herein. In the context of a CDC assay, “target cells” refers to cells used in the CDC assay that express an antigen to which the test molecule can bind. Examples of target cells include WIL2-S, A431, CHO-M7, and BT474. “Test molecule” refers to a molecule that is able to bind the target antigen expressed on target cells and complement protein (e.g. C1q). Examples of types of molecules that can be test molecules include antibodies and heterodimeric molecules.

As used herein, an “antibody” is an immunoglobulin molecule comprising 2 HCs and 2 LCs interconnected by disulfide bonds. The amino terminal portion of each LC and HC includes a variable region of about 100-120 amino acids primarily responsible for antigen recognition via the CDRs contained therein. The CDRs are interspersed with regions that are more conserved, termed framework regions (“FR”). Each LCVR and HCVR is composed of 3 CDRs and 4 FRs, arranged from amino-terminus to carboxy-terminus in the following order: FR1, CDR1, FR2, CDR2, FR3, CDR3, FR4. The 3 CDRs of the LC are referred to as “LCDR1, LCDR2, and LCDR3,” and the 3 CDRs of the HC are referred to as “HCDR1, HCDR2, and HCDR3.” The CDRs contain most of the residues which form specific interactions with the antigen. The functional ability of an antibody to bind a particular antigen is, thus, largely influenced by the amino acid residues within the six CDRs. In a general sense, CDC is typically higher with IgG1 or IgG3 molecules and lower with IgG2 and IgG4 molecules.

As used herein, a heterodimeric molecule is able to bind two or more different antigens or two or more distinct epitopes on the same antigen. A heterodimeric molecule may be used interchangeably with a multispecific antibody. A non-limiting example of a heterodimeric molecule is a bispecific antibody. Examples of bispecific antibody formats can be found in, for example, Labrijn et al., Nat. Rev. Drug Disc.; 18, 585-608 (2019).

An antibody or heterodimeric molecule may bind complement via the Fc region of the antibody or heterodimeric molecule. The Fc region is mainly responsible for binding cellular receptors and/or complement. The Fc region is therefore responsible for antibody effector function and initiation of the complement cascade.

As used herein, “complement” refers to proteins that circulate in blood that can become activated in responses such as an immune response. Examples of complement proteins include C1, C2, C3, C4, C5, C6, C7, C8, and C9. Complement can be human complement, rabbit complement, or another type of complement available to purchase or make by known methods. Complement may be from one of a variety of species, such as rabbit, baby rabbit, human, goat, llama, or guinea pig. In preferred embodiments, complement is tissue culture grade complement, which is bacteria-free, mycoplasma-free, virus-free, and low endotoxin. Human complement refers to complement proteins obtained from human serum. Rabbit complement refers to complement proteins obtained from rabbit serum. In some embodiments, the rabbit complement is obtained from baby rabbit serum. Complement can be purchased from vendors or other commercially available sources. Complement may be lyophilized and then reconstituted in solution, or it may be prepared in solution, such as a frozen solution in a vial.

In a CDC assay, complement can be added either manually or by an automated machine such as the Tecan Freedom EVO® automated liquid handling system programmed to add complement at a desired speed, according to the manufacturer's instructions. In addition, electronic pipettes can be configured to add complement at a desired speed, such methods can be determined from the relevant user manual.

Cell killing (also referred to as cell death or cytotoxicity) in a CDC assay can be measured by methods known to a person of ordinary skill, including, but not limited to, CellTiter-Glo®, detecting the release of cell components during cell death such as GAPDH via fluorescent or luminescent determination, staining and detecting dead cells, adding alamar blue that is converted into a fluorescent molecule only in live cells, and radioactive assays such as measuring radioactive compounds that are released from cells during cell death. Tetrazolium compounds can also be used to detect viable cells. Examples of tetrazolium compounds include MTT, MTS, XTT, and WST-1.

### EXAMPLES

### Example 1: Speed of Complement Addition in CDC Assays

To determine if the speed of complement addition impacted non-specific cell killing and variability, a liquid handling program script was written for the Tecan Freedom EVO® automated liquid handling system. The script added 50 μL of assay media and 50 μL of cells (from two cell banks (Cell Bank A and Cell Bank B) derived from WIL2S cells) at 1.65 cells/mL in bulk to three 96-well assay plates. Next, it was programmed to add 50 μL of either BRC or assay media to each of the three plates at five different speeds, one column at a time. Each speed was programmed into the script through the Dispense Speed parameter of the Liquid Class. Five Liquid Classes were created to dispense at the following speeds: 10 μL/second, 50 μL/second, 100 μL/second, 200 μL/second, or 400 μL/second.

Assay media or baby rabbit complement (“BRC”) were also added manually to a column of each plate using a Rainin XLS™ multichannel electronic pipette set to a dispense speed of 9 (out of 10). The plates were incubated for 45-75 minutes at 37° C. and 5% CO₂. After a 5-10 minute cool-down, 50 μL of CellTiter-Glo® (Promega) reagent was added to all wells of the plates. After a 15-30 minute incubation, the plates were read on an EnVision® plate reader, measuring luminescence. The difference in signal between the wells containing cells and media and the wells containing cells, complement, and media is a measure of the non-specific killing of the cells by the complement. A higher ratio indicates more nonspecific cell killing. Results are shown in Table 1.

TABLE 1
Effect of the Speed of Addition of BRC on the Cell Viability Ratio.

| | |
|---|---|
| Speed of | Ratio (Cells and Media/Cells, |
| Complement + Media or | Media, and Complement) |

| Media Alone Addition | Cell Bank A | Cell Bank B |
|---|---|---|
| Speed 1 (50 μL/s) | 4.6 | 7.1 |
| Speed 2 (10 μL/s) | 5.1 | 6.9 |
| Speed 3 (100 μL/s) | 4.1 | 5.5 |
| Speed 4 (200 μL/s) | 2.0 | 2.4 |
| Speed 5 (400 μL/s) | 1.4 | 1.4 |
| Manual Complement | 1.5 | 1.4 |
| Addition (429 μL/s) | | |

These data demonstrate that BRC speed of addition is the critical factor for non-specific killing, and that higher speeds of complement addition result in reduced non-specific cell killing.

### Example 2: Sources of Complement in CDC Assays

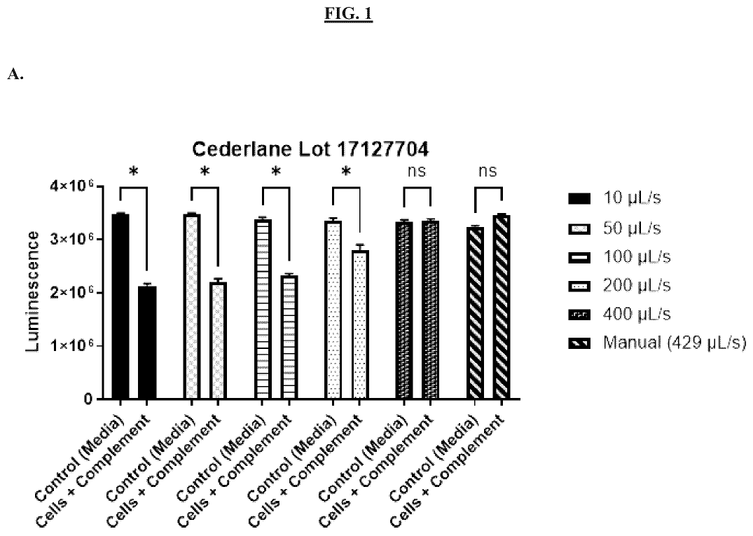

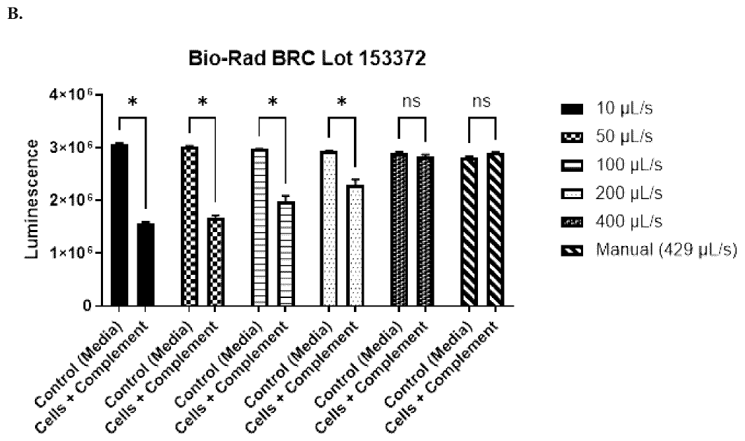

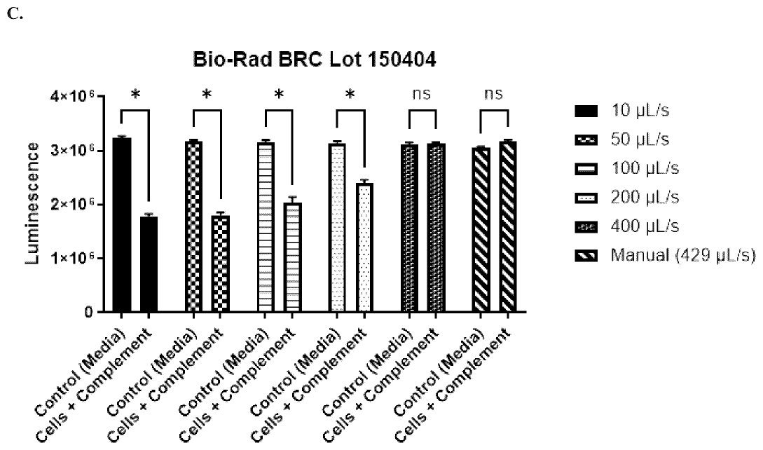

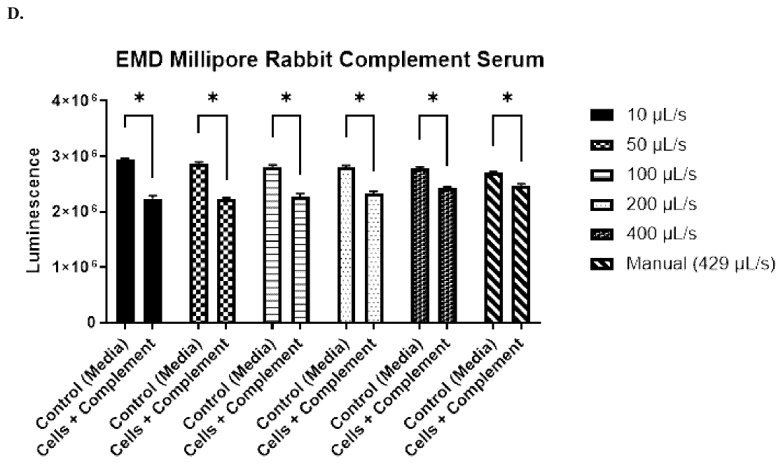

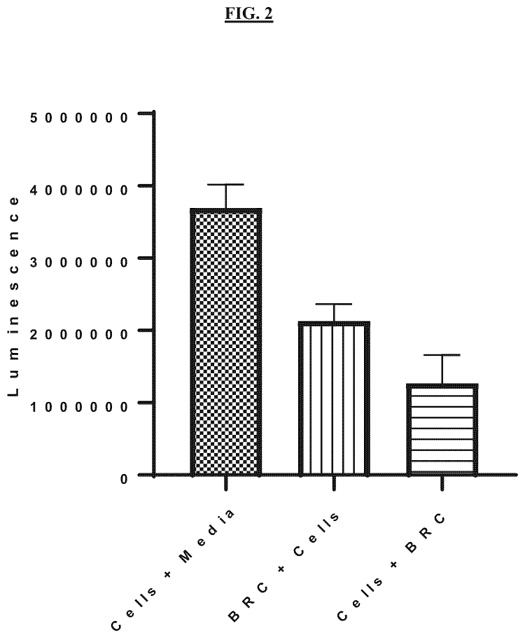

Other sources of BRC were also studied to determine if different speeds of addition impacted non-specific cell killing. Cederlane BRC (cat #CL3441-S100) from a different lot used in Example 1, two lots from Bio-Rad (cat #C12CA), and one lot of BRC from EMD Millipore (cat #234400) were tested. The study was performed essentially as described in Example 1, except four plates were prepared instead of three plates, and each plate used a different lot of one of the four lots of complement. The Cederlane lot was diluted (1:16.5), while the other three lots were diluted 1:12 based off previously qualified concentrations. The results are shown in FIG. 1.

These data demonstrate that similar to the data in Example 1, the speed of addition of other lots and sources of complement (Cederlane lots and two Bio-Rad lots) impacted the cell viability ratios of WIL2-S cells. Specifically, non-specific cell killing was reduced when complement was added at a faster speed. However, speed of addition was not a factor for EMD Millipore lot of Rabbit complement at the dilution tested (1:12).

In another experiment, the speed of addition of the EMD Millipore Rabbit Complement Serum did not appear to have an impact on the ratio of the controls at dilutions of 1:2, 1:15, and 1:30 in WIL2-S cells or CHO-M7 cells.

### Example 3: Order of Addition of BRC

To determine if adding BRC to the plate before or after cells impacts non-specific cell killing and assay variability, a preparation of product dilutions, cells (WIL2-S), and BRC (purchased from Cederlane) were prepared. For this assay, product dilutions were added, followed by BRC, and then followed by cells. Less than about one minute elapsed between the addition of each of the components, and BRC was added at a speed of 105 μL/second. Plates were incubated at 37° C. at 5% CO₂ for 60 minutes. Cell killing was determined using Cell Titer Glo. The cell viability ratio following these procedures was 0.94, indicating relatively low levels of non-specific cell killing when BRC is added to the plate before cells are added.

Without being bound by theory, these data suggest that when cells are first added to a plate, followed by addition of BRC, a gradient of BRC forms, resulting in non-specific cell killing. This non-specific killing can be reduced either by adding complement at a higher speed (e.g. 250 μL/s to about 500 μL/s), or adding BRC to the plate first, followed by addition of cells.

In another experiment, BRC was added to the plate before or after addition of cells to analyze non-specific cell killing. In a 24-well plate, WIL2-S cells were added first, followed by addition of BRC (Cederlane). In another 24-well plate, BRC was added first, followed by addition of cells. Nonspecific killing of the WIL2-S cells was calculated by comparing the ratio of the mean of treated wells to the mean of control wells containing only cells and media. The closer that ratio is to 1 the less nonspecific killing there is in the assay. As shown in Table 2 and FIG. 1, there is less nonspecific killing of WIL2-S cells when the BRC is added first.

Therefore, adding BRC before adding the cells results in more reliable assay results.

TABLE 2
The Effect of Order of Addition of BRC and
Cells on the Control Ratio of WIL2-S Cells.

| | Ratio (Cells + media/Cells + |
|---|---|
| Order of Addition | Complement) |
| Cells first | 0.34 |
| BRC first | 0.56 |

1. A method of performing a complement-dependent cytotoxicity (CDC) assay comprising adding complement to a mixture of target cells and test molecule, wherein the complement is added at a speed of at least 250 μL/s.

2. The method of claim 1, wherein the complement is added at a speed of at least about 400 μL/s.

3. The method of claim 1, wherein the complement is added at a speed of about 400 μL/s.

4. The method of claim 1, wherein the complement is added at a speed of about 450 μL/s.

5. The method of claim 1, wherein the complement is added at a speed of about 500 μL/s.

6. The method of claim 1, wherein the complement is added at a speed of at least about 500 μL/s.

7. The method of claim 1, wherein the complement is baby rabbit complement.

8. The method of claim 7, wherein the complement is from Bio-Rad or Cedarlane.

9. The method of claim 7, wherein the complement is not from EMD.

10. A method of performing a CDC assay comprising adding target cells and test molecule to a mixture comprising complement and media.

11. The method of claim 10, wherein the complement is baby rabbit complement.

12. A method comprising adding complement to target cells, wherein the method comprises adding complement to the target cells at a speed of at least 250 μL/s.

13. The method of claim 12, wherein the complement is added to the target cells at a speed of at least about 400 μL/s.

14. The method of claim 12, wherein the complement is added to the target cells at a speed of about 400 μL/s.

15. The method of claim 12, wherein the complement is added to the target cells at a speed of about 450 μL/s.

16. The method of claim 12, wherein the complement is added to the target cells at a speed of at least about 500 μL/s.

17. The method of claim 12, wherein the complement is baby rabbit complement.

18. The method of claim 17, wherein the complement is from Bio-Rad or Cedarlane.

19. The method of claim 12, wherein the complement is added to target cells in an assay to determine cell death.

20. The method of claim 19, wherein the assay is a complement-dependent cytotoxicity assay.

21. The method of claim 1, wherein the test molecule is an antibody.

22. The method of claim 1, wherein the test molecule is an IgG1 or IgG3 antibody.

23. The method of claim 1, wherein the test molecule is a heterodimeric molecule.
